(19) United States
(12) Patent Application Publication
(10) Pub. No.: US 2015/0204865 A1
(43) Pub. Date: Jul. 23, 2015

(54) SENSING METHOD

(71) Applicant: SIWARD Crystal Technology Co., Ltd., Taichung (TW)

(72) Inventors: Kuan-Jiuh Lin, Taichung (TW); Chuen-Yuan Hsu, Taichung (TW); Wei-Hung Chen, Taichung (TW); Yi-Heui Hsieh, Taichung (TW); Yun-Ting Chiang, Taichung (TW); Jia Yu Chang, Taichung (TW)

(73) Assignee: SIWARD CRYSTAL TECHNOLOGY CO., LTD., Taichung (TW)

(21) Appl. No.: 14/601,754

(22) Filed: Jan. 21, 2015

(30) Foreign Application Priority Data
Jan. 22, 2014 (TW) 103102349

(51) Int. Cl. G01N 33/553

(52) U.S. Cl. CPC G01N 33/553

(57) ABSTRACT

A sensing method, comprising steps of causing a first molecule to be adjacent to one of a plurality of first nanoparticles spacedly disposed on a detachable chip; adding a target object to contact the first molecule; and measuring a spectral signal, wherein a variation of the spectral signal of the plurality of first nanoparticles occurs when the target object demonstrates a first specific binding with the first molecule.

### CROSS REFERENCE TO RELATED APPLICATIONS

The application claims the benefit of Taiwan Patent Application No. 103102349, filed on Jan. 22, 2014, at the Taiwan Intellectual Property Office, the disclosures of which are incorporated herein in their entirety by reference.

### FIELD OF THE INVENTION

The present invention relates to a sensing method, and more particularly to a sensing method using localized surface plasmon resonance.

### BACKGROUND OF THE INVENTION

Experimental plates on the market have a variety of structures and materials according to different experimental requirements. For example, they can be divided into 6, 12, 24, 48, 96, 384 and 1536 well plates based on the number of the wells, can be divided into a flat bottom, round bottom, V-bottom and Easy-Wash bottom (it has characteristics of both the round well bottom and flat well bottom) plates based on the structure of the bottom, can be divided into polystyrene (PS), polypropylene (PP) and poly(vinyl chloride) (PVC) plates based on the material, can be divided into clear, black, white, black with clear bottom and white with clear bottom plates based on the color, and can be divided into general analysis, cell culture and cell analysis, immunoassay and storage plates based on the use. Most immunoassay plates are polystyrene 96-well plates. The bottom surface of immunoassay plates is usually un-treated or treated to cause benzene rings on the plate surface to produce carboxyl groups and hydroxyl groups to increase the binding capacity with molecules intended to be coated on the plates using irradiation technology.

Enzyme-linked immunosorbent assay (ELISA) is a common sensing method, and it has a history of many years. There are at least two types of ELISA, wherein in one type of ELISA, the target object is an antigen; in another type, the target object is an antibody. They are discussed as follows.

1. When the target object is an antigen, ELISA includes the following steps:

(1) Coating a specific antibody on a plastic plate, wherein the time for coating is about 12-18 hours, and washing away excess antibodies after the completion of the coating;

(2) Adding the target object to carry out a reaction with the coated antibody, wherein the reaction time is about 0.5-2 hours, and if the target object contains an antigen reactive with the coated antibody, the antigen carrying out a specific binding with the coated antibody on the plastic plate;

(3) Washing away excess target objects, and then adding an antibody with an enzyme reactive with the antigen to bind with the antigen, wherein the time for binding is about 0.5-2 hours; and

(4) Washing away excess un-bound antibodies with an enzyme, adding a substrate for the enzyme to carry out a color reaction, wherein the time for the color reaction is about 0.5 hour, and then reading the result of the color reaction (i.e. absorbance (OD value)), wherein it takes about 1-2 days to complete the entire experiment.

2. When the target object is an antibody, ELISA includes the following steps:

(1) Coating a known antigen on a plastic plate, wherein the time for coating is about 12-18 hours, and washing away excess antigens after the completion of the coating;

(2) Adding the target object to carry out a reaction with the coated antigen, wherein the reaction time is about 0.5-2 hours, and if the target object contains a first antibody reactive with the coated antigen, the first antibody carrying out a specific binding with the coated antigen on the plastic plate;

(3) Washing away excess target objects, and then adding a second antibody with an enzyme to bind with the first antibody, wherein the time for binding is about 0.5-2 hours; and

(4) Washing away excess un-bound second antibodies with an enzyme, adding a substrate for the enzyme to carry out a color reaction, wherein the time for the color reaction is about 0.5 hour, and then reading the result of the color reaction (i.e. absorbance (OD value)), wherein it takes about 1-2 days to complete the entire experiment.

The plate used for ELISA is an immunoassay plate. No matter whether the plate is un-treated or treated with the irradiation technology, the step of coating an antibody or antigen on the plate is performed by physical adsorption, which is non-specific. Thus, the step for coating takes 12-18 hours of reaction time, and ELISA further includes the reaction time of an antibody with an enzyme reacting with a substrate for the enzyme, so that the entire experiment take 1-2 days to be completed. In addition, an expensive antibody with an enzyme and the substrate for the enzyme are needed for ELISA. Therefore, ELISA has room for improvement in both the time required and the price.

Surface plasmon resonance (SPR) is a sensing technology developed in recent years. The principle of SPR is that when an external light source irradiates a metal film having a nanostructure at any angle, if there is a wavelength the same as the resonance wavelength of the free electrons on the metal surface, the free electrons will be excited to vibrate collectively and cause the absorbance of light to generate the wavelength λ1. If a bonding is formed between the metal surface and a biological or chemical molecule, the wavelength λ1 will shift to λ2. By detecting changes of the wavelength, the nature and concentration of the target object will be known. SPR requires a shorter time than ELISA, but needs dedicated instruments, so its price is more expensive, and its implementation is relatively inconvenient.

Localized surface plasmon resonance (LSPR) is developed after SPR, and it is a superior technique. The principle of LSPR is that when metal nanoparticles are fabricated on a transparent substrate, the excitation of the incident light will cause nanoparticles surface to produce surface plasmonic resonance. Because the frequency and intensity of the resonance are susceptible to the surrounding environment and produce a wavelength shift or a variation of intensity of a signal, the change of the local dielectric constant can be used to detect the analyte. As long as there are analytes binding adjacent to the particles, an optical variation can be measured by an optical instrument. The surface of a nanoparticle is like a tiny detector, and a high optical signal can be measured in the range of several nanometers.

The main difference between LSPR and SPR is the distance from the surface on which the respective plasmons can detect changes. The penetration depth of the plasmon field for SPR is between 200-1000 nm, but that for LSPR is only 15-30 nm. Hence, LSPR is far less susceptible to bulk effects occurring away from the surface. In other words, LSPR detects changes very close to the surface, thereby allowing compatibility with a complex or crude reaction solution.

Table 1 shows a comparison of three sensing systems for inter molecular recognition, which include ELISA, SPR and LSPR. It can be seen from Table 1 that the performance of LSPR is very good for every item. Compared to ELISA, LSPR can be label-free and real-time detection; compared to SPR, LSPR does not need temperature control. The cost of LSPR is lower than both ELISA and SPR. However, there are still many problems to be solved to commercialize LSPR.

TABLE 1
Comparison of ELISA, SPR and LSPR

| Feature/characteristic | ELISA | SPR | LSPR |
|---|---|---|---|
| Label-free detection | No | Yes | Yes |
| Detection modes | Optical | Angle shift | Extinction change |
| | absorbance | or wavelength | or wavelength |
| | | shift | shift |
| Temperature control | No | Yes | No |
| Non-specific binding | Minimal | Minimal | Minimal |
| Small molecule | Good | Good | Better |
| sensitivity | | | |
| Real-time detection | No | Yes | Yes |
| Microfluidics | No | Yes | Yes |
| compatibility | | | |
| Cost | Expensive | Expensive | Cheap |
| Field portability | No | No | Yes |
| Commercial | Yes | Yes | Not popular |
| potential | | | |

Currently, LamdaGen is the only LSPR product on the market. The principle of LamdaGen includes providing a substrate surface with three-dimensional structures such as undulating creases, porous materials and nanowires; depositing nanoparticles such as gold or silver nanoparticles on the substrate surface with three dimensional structures to serve as LSPR sensing materials; attaching capture agents such as DNA or IgG to the surfaces of the nanoparticles; and providing an incident light to the substrate, collecting a reflected light, monitoring the kinetics, and quantitating an analyte concentration by reading a wavelength shift via a spectrometer. LamdaGen's products only can be read by LamdaGen's instruments, which are expensive and cause a great burden for the user.

Thus, the sensing methods available on the market still have many problems to be solved such as the time required and the price. Although LSPR is a sensing method having many advantages and there is an LSPR product on the market, it is not popular because it is expensive and inconvenient. To make LSPR more popular, the price and convenience of use are the problems to be urgently solved.

### SUMMARY OF THE INVENTION

In accordance with one aspect of the present invention, a sensing method is disclosed. The sensing method includes steps of providing a detachable chip including a substrate and a nanoparticle unit, wherein the substrate is made of a transparent material and the nanoparticle unit is arranged on the substrate and includes a plurality of nanoparticles spacedly disposed on the substrate; providing a base element with a through hole, and disposing the detachable chip detachably on the base element to close the through hole at one end of the base element to form a complex sensing element; providing a frame disposing therein the complex sensing element; disposing a first molecule to be adjacent to one of the plurality of nanoparticles; adding a target object to the complex sensing element to initiate a first specific binding between the first molecule and the target object; and disposing the complex element in a spectrometer to obtain a value of a spectral signal of the plurality of spaced nanoparticles.

In accordance with another aspect of the present invention, a sensing device is disclosed. The sensing device includes a detachable chip; a base element having a through hole, wherein the detachable chip detachably arranged on the base element to close the through hole at one end of the base element to form a complex sensing element; and a frame disposing therein the complex sensing element.

In accordance with a further aspect of the present invention, a sensing device is disclosed. The sensing method includes steps of causing a first molecule to be adjacent to one of a plurality of first nanoparticles spacedly disposed on a detachable chip; and adding a target object to contact the first molecule; and measuring a spectral signal, wherein a variation of the spectral signal of the plurality of first nanoparticles occurs when the target object demonstrates a first specific binding with the first molecule.

### BRIEF DESCRIPTION OF THE DRAWINGS

FIG. 1(a) shows the detachable chip according to a first embodiment of the present invention;

FIG. 1(b) shows the base element with a through hole according to the first embodiment of the present invention;

FIG. 1(c) shows the complex sensing element according to the first embodiment of the present invention;

FIG. 2(a) shows the detachable chip according to a second embodiment of the present invention;

FIG. 2(b) shows the base element with a through hole according to the second embodiment of the present invention;

FIG. 2(c) shows the complex element according to the second embodiment of the present invention;

FIG. 3 shows the frame of the present invention;

FIG. 4(a) shows that 6 sets of complex elements 23 are used when the number of samples is 48;

FIG. 4(b) shows that 12 sets of complex sensing elements 23 are used when the number of samples is 96;

FIG. 5(a) shows the detachable chip according to a third embodiment of the present invention;

FIG. 5(b) shows the base element with a through hole according to the third embodiment of the present invention;

FIG. 5(c) shows the complex sensing element according to the third embodiment of the present invention;

FIG. 6(a) shows the detachable chip according to a fourth embodiment of the present invention;

FIG. 6(b) shows the sensing chip carrier according to the fourth embodiment of the present invention;

FIG. 6(c) shows the sensing chip carrier according to the fourth embodiment of the present invention;

FIG. 7 shows the coating characteristics of the substrate with microwave plasma nanoparticles of the present invention;

FIG. 8 shows the production and detection of reproducibility of the sensing chip of the present invention;

FIG. 9(a) shows the structural stability test for the sensing chip of the present invention;

FIG. 9(b) shows the surface oxidation effect test for the sensing chip of the present invention;

FIG. 10 shows the method of further signal amplification for the sensing chip of the present invention; and

FIG. 11 shows the sensing method of the present invention.

### DETAILED DESCRIPTION OF THE PREFERRED EMBODIMENTS

The present invention will now be described more specifically with reference to the following embodiments. It is to be noted that the following descriptions of preferred embodiments of this invention are presented herein for the purposes of illustration and description only; they are not intended to be exhaustive or to be limited to the precise form disclosed.

A preferred embodiment of the present invention is a sensing method, which is used for qualitative and quantitative research of a target object.

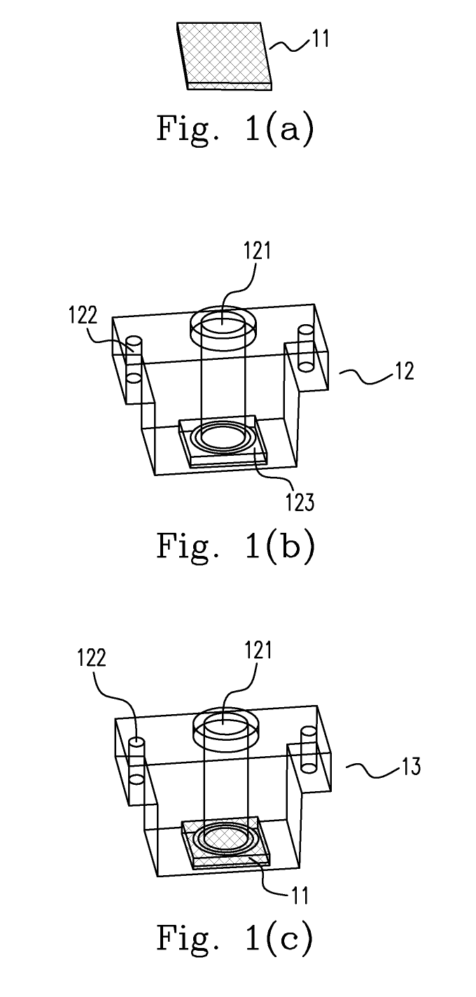

As shown in FIGS. 1(a)-3, the sensing method includes the following steps. A detachable chip 11 is provided, wherein the area of the detachable chip 11 is 1-49 mm². For example, it may be (1-7 mm)*(1-7 mm). The shape of the detachable chip 11 is one selected from a group consisting of a circle, ellipse, polygon, irregular shape, and a combination thereof. The detachable chip 11 includes a substrate and a nanoparticle unit, wherein the substrate is made of a transparent material, which is one selected from a group consisting of polyethylene (PE), High-density polyethylene, Low-density polyethylene, polypropylene (PP), polystyrene (PS), poly(vinyl chloride) (PVC), Polyethylene terephthalate (PET), poly(dimethylsiloxane) (PDMS), Polymethylmethacrylate (PMMA), Polycarbonates (PC), glass, quartz, quartz glass, mica, sapphire, transparent ceramic, and a combination thereof. The nanoparticle unit is arranged on the substrate and includes a plurality of first nanoparticles spacedly disposed on the substrate. The manufacturing method for the first nanoparticles can be that disclosed in Taiwan Patent No. I404930. The first nanoparticles are made of a metal which is one selected from a group consisting of gold (Au), silver (Ag), palladium (Pd), platinum (Pt), chromium (Cr), cobalt (Co), molybdenum (Mo), copper (Cu), nickel (Ni), aluminum (Al), iron (Fe), magnesium (Mg), tin (Sn), titanium (Ti), thallium (Ta), iridium (Ir), an alloy thereof, and a combination thereof. The shape of the nanoparticles is one selected from a group consisting of a circle, island, long strip, triangle, star, annulus, hollow shape, and a combination thereof. The diameter of the nanoparticles is 1-200 nm. There is a distance between the nanoparticles, and the distance is 1-100 nm.

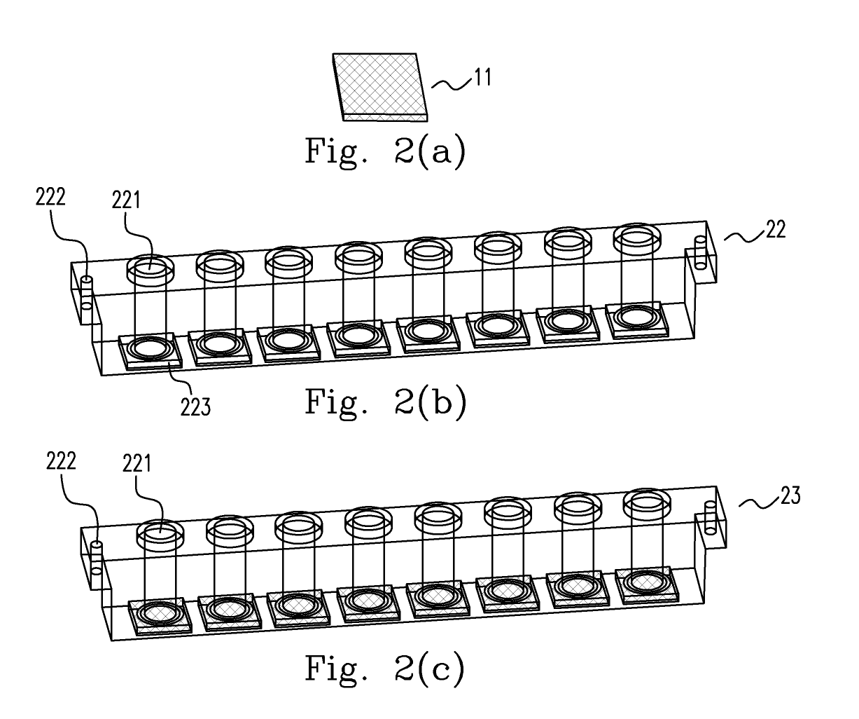

A base element with a through hole 12, 22 is provided (Please refer to FIG. 1(b) and FIG. 2(b)), wherein the base element with a through hole 12, 22 has a through hole 121, 221 and a scarfing hole 122, 222. The number of the through holes of the base element with a through hole 12, 22 is an integer between 1-384. The detachable chip 11 is detachably arranged on the base element 12, 22 to close the through hole 121, 221 at one end of the base element 12, 22 to form a complex sensing element 13, 23. One end of the base element 12, 22 may have a recess 123, 223, and the detachable chip 11 may be arranged in the recess 123, 223 to form a complex sensing element 13, 23. The connection method for the detachable chip 11 and the base element with a through hole 12, 22 may include but is not limited to bonding, riveting, screwing, welding, scarfing and articulating.

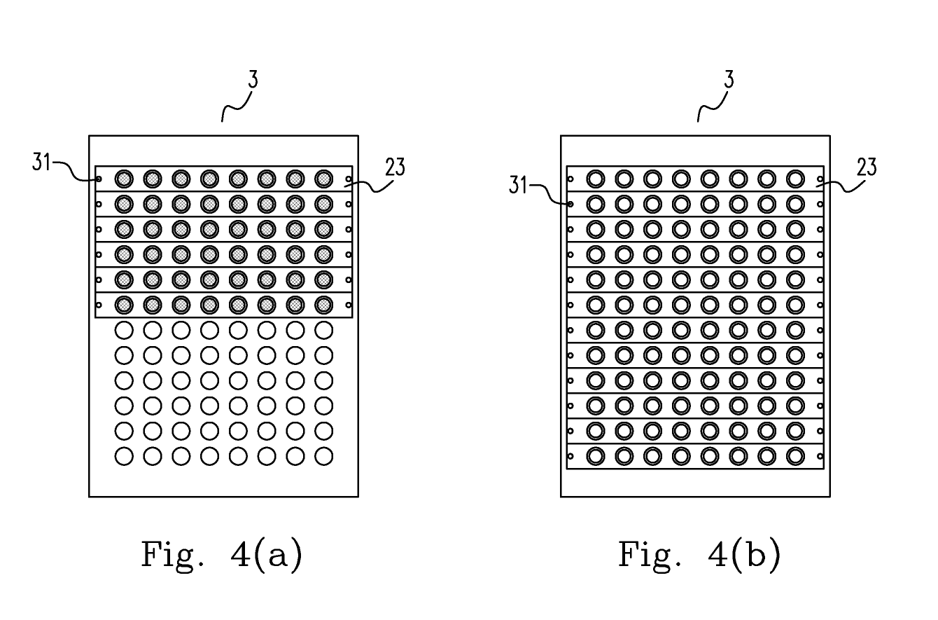

A frame 3 is provided, wherein the complex sensing element 13, 23 is disposed therein for sensing, and the disposing method may include but is not limited to bonding, riveting, screwing, welding, scarfing and articulating. The disposing method in this embodiment is scarfing. The frame 3 has a scarfing column 31 to combine the scarfing hole 122 of the base element with a through hole 12, 22 for use. The number of the through holes of the base element 12, 22 and the number of the set of the complex sensing element 13, 23 may depend on the user's needs. As shown in FIG. 4(a) and FIG. 4(b), when the number of samples is 48, 6 sets of the complex sensing element 23 with the base element having 8 through holes 22 may be used. When the number of samples is 96, 12 sets of the complex sensing element 23 with the base element having 8 through holes 22 may be used. Unlike conventional 96-well plates, regardless of the number of samples, an entire 96-well plate is used. On the other hand, when the number of samples is large but the quantity of the individual sample is small, or when samples are expensive, 1 set of the complex sensing element with the base element having 384 through holes may be used. Accordingly, a large number of samples may be handled at a time, and the amount of samples may be saved.

A first molecule is disposed adjacent to one of the plurality of nanoparticles. The manufacturing method can be that disclosed in Taiwan Patent No. I404930. The first molecule is determined to be disposed on the surface of the substrate according to the type of the target object to be screened. When the first molecules form a specific binding with the target objects, the localized electromagnetic field induced by the illumination of the metal nanoparticles will change due to the affect of the surrounding environment, which leads to a variation of the spectral signal. Thus, researchers are able to take advantage of the variation of the spectral signal of the metal nanoparticles before and after the first molecules bind with the target objects to detect whether a sample contains the target object and then quantify its concentration. This allows the sensing method of the present invention to have both qualitative and quantitative characteristics. For example, when the target object is streptavidin, because streptavidin and biotin can form a specific binding, biotin can be used as the first molecule. Because biotin can not form a stable binding with the substrate directly, (3-Aminopropyl)trimethoxysilane (APTMS), which can bind with the substrate more easily and can form a bonding with biotin, can be used. First, an APTMS molecule membrane is formed on the substrate surface, and then biotin is added, such that biotin can be deposed on the substrate surface indirectly through APTMS. The combination of APTMS and biotin is the first molecule. In addition, when the target object is mercurous ion, because mercurous ion and 4-carboxybenzo-15-crown-5-ether can form a specific binding, 4-carboxybenzo-15-crown-5-ether can be used as the first molecule. First, saline is deposited on the substrate surface, and then 4-carboxybenzo-15-crown-5-ether is linked, such that mercurous ion can be detected. In this case, the combination of saline and 4-carboxybenzo-15-crown-5-ether is the first molecule. The above manner of chemically modifying a substrate using an appropriate molecule can shorten the time for coating the first molecule to the substrate to only one hour. Compared to ELISA, which takes 12-18 hours to coat an antigen or antibody to the substrate, the above manner is substantially faster.

A target object is added to the through hole 121, 221 of the complex sensing element 13, 23 to contact the disposed first molecule. A variation of the spectral signal of the plurality of first nanoparticles occurs when the target object demonstrates a first specific binding with the first molecule, wherein the variation of the spectral signal is caused by localized surface plasmon resonance (LSPR).

The complex sensing element disposed in the frame 3 is disposed in a spectrometer to obtain a value of the variation. The value is a wavelength, and the read wavelength ranges from 300-700 nm. The read wavelength ranges will vary with the diameter of the metal nanoparticles (or the thickness of the metal layer) and the material of the metal nanoparticles. For example, when the average diameter of the metal nanoparticles is 5 nm-20 nm, the read wavelength will range from 400 nm-650 nm. When the total thickness of the metal layer is controlled at 3 nm, the formed gold nanoparticles will mainly range from 510 nm-540 nm, and the formed gold and silver alloy nanoparticles will mainly range from 410 nm-490 nm. The device for sensing in the present invention has the same length and width as general experimental plates on the market, and therefore it is suitable for any instrument using general experimental plates such as a spectrometer and an automatic microplate washer, wherein the spectrometer may be an ELISA reader.

The sensing method in the present invention may further include the step of adding a second nanoparticle labeled with a second molecule to initiate a second specific binding with the target object. The second molecule may be but is not limited to an antigen or antibody. The second specific binding will amplify the variation of the spectral signal.

Another preferred embodiment of the present invention is a sensing method including the following steps. A first molecule is caused to be adjacent to one of a plurality of first nanoparticles spacedly disposed on a detachable chip. A target object is added to contact the first molecule, and a spectral signal of the plurality of first nanoparticles is measured, wherein a variation of the spectral signal of the plurality of first nanoparticles occurs when the target object demonstrates a first specific binding with the first molecule. The spectral signal may be measured by a microplate spectrometer, and the variation of the spectral signal comes from localized surface plasmon resonance (LSPR). The first nanoparticles have a diameter ranged from 1-200 nm, and every two adjacent first nanoparticles have a distance ranged from 1-100 nm. The first nanoparticles are made of a metal which is one selected from a group consisting of gold (Au), silver (Ag), palladium (Pd), platinum (Pt), chromium (Cr), cobalt (Co), molybdenum (Mo), copper (Cu), nickel (Ni), aluminum (Al), iron (Fe), magnesium (Mg), tin (Sn), titanium (Ti), thallium (Ta), iridium (Ir), an alloy thereof, and a combination thereof. The sensing method in this embodiment is used for at least one of qualitative and quantitative research of the target object. The sensing method in this embodiment may further include the step of adding a second nanoparticle labeled with a second molecule to initiate a second specific binding with the target object so as to amplify the variation in the spectral signal.

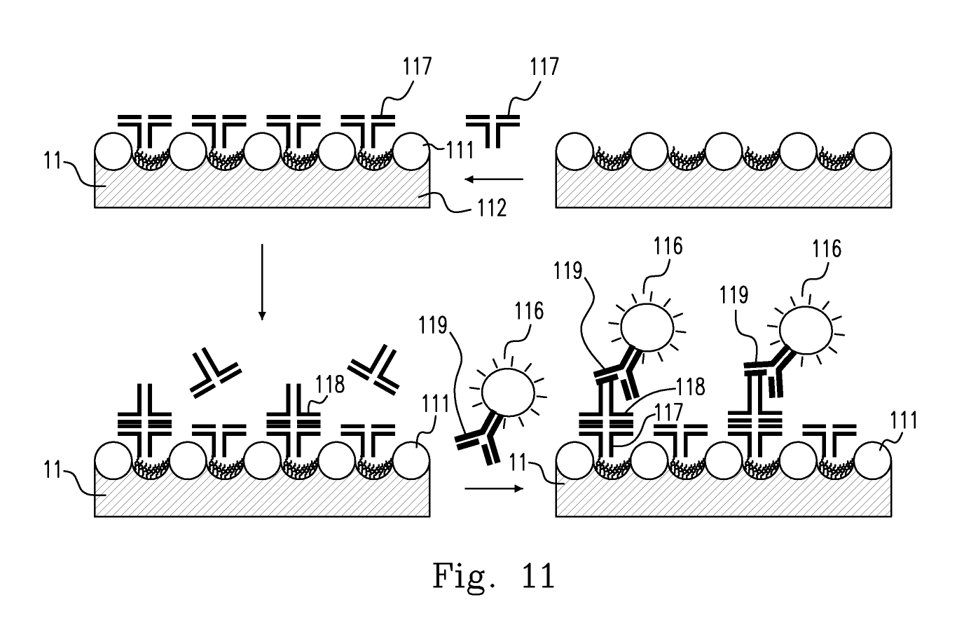

Another preferred embodiment of the present invention is a sensing method. As shown in FIG. 11, the sensing method includes the following steps. A detachable chip 11 is provided. The detachable chip 11 includes a substrate and a nanoparticle unit, wherein the substrate is made of a transparent material, and the nanoparticle unit is arranged on the substrate and includes a plurality of first nanoparticles spacedly disposed on the substrate. The nanoparticles are made of a metal which is one selected from a group consisting of gold (Au), silver (Ag), palladium (Pd), platinum (Pt), chromium (Cr), cobalt (Co), molybdenum (Mo), copper (Cu), nickel (Ni), aluminum (Al), iron (Fe), magnesium (Mg), tin (Sn), titanium (Ti), thallium (Ta), iridium (Ir), an alloy thereof, and a combination thereof. A base element with a through hole 12, 22 is provided, wherein the detachable chip 11 is detachably arranged on the base element 12, 22 to close the through hole 121, 221 at one end of the base element 12, 22 to form a complex sensing element 13, 23. A frame 3 is provided, wherein the complex sensing element 13, 23 is disposed therein for sensing. A first molecule 117 is disposed adjacent to one of the plurality of nanoparticles. A target object is added to the through hole 121, 221 of the complex sensing element 13, 23 to initiate a first specific binding between the first molecule and the target object. A second molecule 119 labeled with a luminant molecule 116 is added to initiate a second specific binding between the target object 118 and the second molecule 119. The luminescent molecule 116 may be a fluorescent molecule or a luminescent molecule. When the luminant molecule 116 is a fluorescent molecule, the fluorescent molecule may be but is not limited to Fluorescein isothiocyanate (FITC), phycoerythrin (PE), Allophycocyanin (APC), or Peridinin chlorophyll protein (PerCP). When the luminant molecule 116 is a luminescent molecule, the luminescent molecule may be a bioluminescent molecule or a chemiluminescent molecule. The second molecule 119 may be but is not limited to an antigen or an antibody. When the target object 118 exhibits the first specific binding with the first molecule 117 and the second molecule 119 exhibits the second specific binding with the target object 118, the nanoparticles have localized surface plasma resonance. The localized surface plasmon resonance generates a localized electromagnetic field, causes a strong electromagnetic coupling effect to be generated between the luminant molecule 116 and the nanoparticles, increases the luminant intensity of the luminant molecule, and improves the sensitivity for detecting the target object 118. The complex sensing element is disposed in a spectrometer to obtain a value of a spectral signal of the plurality of spaced nanoparticles. In this embodiment, due to the electromagnetic coupling effect generated between the luminant molecule 116 and the nanoparticles, the value rises substantially. This causes the reaction sensitivity to significantly increase.

Another preferred embodiment of the present invention is a sensing device used for at least one of qualitative and quantitative research of a target object, wherein the target object is one selected from a group consisting of a protein, a cell, a compound, a metal ion, and a combination thereof.

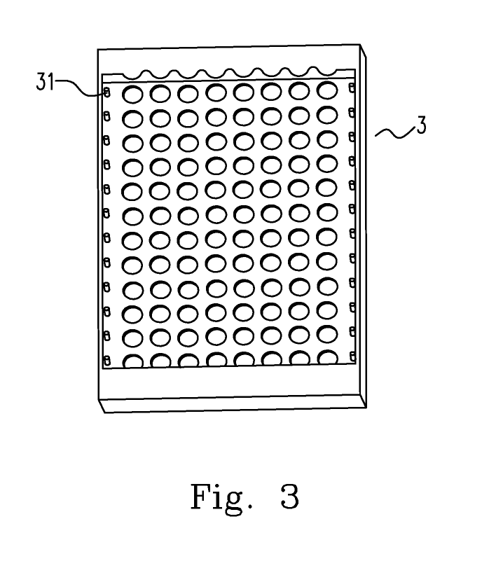

As shown in FIGS. 1(a)-3, the sensing device includes a detachable chip 11, a base element with a through hole 12, 22 (Please refer to FIG. 1(b) and FIG. 2(b)) and a frame 3 (Please refer to FIG. 3). The area of the detachable chip 11 is 1-49 mm². For example, it may be (1-7 mm)*(1-7 mm). The shape of the detachable chip 11 is one selected from a group consisting of a circle, ellipse, polygon, irregular shape, and a combination thereof. The detachable chip 11 includes a substrate, a nanoparticle unit and a sensing unit, wherein the substrate is made of a transparent material, which is one selected from a group consisting of polyethylene (PE), High-density polyethylene, Low-density polyethylene, polypropylene (PP), polystyrene (PS), poly(vinyl chloride) (PVC), Polyethylene terephthalate (PET), poly(dimethylsiloxane) (PDMS), Polymethylmethacrylate (PMMA), Polycarbonates (PC), glass, quartz, quartz glass, mica, sapphire, transparent ceramic, and a combination thereof. The nanoparticle unit is arranged on the substrate and includes a plurality of nanoparticles separately disposed on the substrate. The manufacturing method of the nanoparticles can be the method disclosed in Taiwan Patent No. I404930. The nanoparticles are made of a metal which is one selected from a group consisting of gold (Au), silver (Ag), palladium (Pd), platinum (Pt), chromium (Cr), cobalt (Co), molybdenum (Mo), copper (Cu), nickel (Ni), aluminum (Al), iron (Fe), magnesium (Mg), tin (Sn), titanium (Ti), thallium (Ta), iridium (Ir), an alloy thereof, and a combination thereof. The shape of each nanoparticle is one selected from a group consisting of a circle, island, long strip, triangle, star, annulus, hollow shape, and a combination thereof. The diameter of each nanoparticle is 1-200 nm. Every two adjacent first nanoparticles have a distance therebetween ranged from 1-100 nm.

The sensing unit includes a plurality of receptors disposed adjacent to one of the plurality of nanoparticles. The manufacturing method can be the method disclosed in Taiwan Patent No. I404930. The receptor disposed on the surface of the substrate is determined according to the kind of target object to be screened. When the receptors of the sensing unit form a specific binding with the target objects, the localized electromagnetic field induced by illuminating the metal nanoparticles will change due to the affect of the surrounding environment, which leads to a variation of the spectral signal. Thus, researchers are able to take advantage of the variation of the spectral signal of the metal nanoparticles before and after the receptors bind with the target objects to detect whether a sample contains the target object and then quantify its concentration. This allows the sensing device of the present invention to have both qualitative and quantitative characteristics. For example, when the target object is streptavidin, because streptavidin and biotin can form a specific binding, biotin can be used as the receptor. Because biotin can not form a stable binding with the substrate directly, (3-Aminopropyl)trimethoxysilane (APTMS), which can bind with the substrate more easily and can form a bonding with biotin, can be used. First, an APTMS molecule membrane is formed on the substrate surface, and then biotin is added, such that biotin can be disposed on the substrate surface indirectly through APTMS. The combination of APTMS and biotin is the receptor. In addition, when the target object is mercurous ion, because mercurous ion and 4-carboxybenzo-15-crown-5-ether can form a specific binding, 4-carboxybenzo-15-crown-5-ether can be used as the receptor. First, saline is deposed on the substrate surface, and then 4-carboxybenzo-15-crown-5-ether is linked, such that the mercurous ion can be detected. In this case, the combination of saline and 4-carboxybenzo-15-crown-5 is the receptor. The above manner of chemically modifying a substrate using an appropriate molecule can shorten the time of coating the receptor on the substrate to only one hour. Compared to ELISA, which takes 12-18 hours to coat an antigen or antibody on the substrate, the above manner is substantially faster.

The base element with a through hole 12, 22 has a through hole 121, 221 and a scarfing hole 122, 222. The number of the through holes of the base element with a through hole 12, 22 is an integer between 1-384. The detachable chip 11 is detachably arranged on the base element 12, 22 to close the through hole 121, 221 at one end of the base element 12, 22 to form a complex sensing element 13, 23. One end of the base element 12, 22 may have a recess 123, 223, and the detachable chip 11 may be arranged in the recess 123, 223 to form a complex sensing element 13, 23. The connection method of the detachable chip 11 and the base element with a through hole 12, 22 may include but is not limited to bonding, riveting, screwing, welding, scarfing and articulating. The complex sensing element 13, 23 is used to contain the target object such that the target object contacts the surface of the detachable chip 11 directly, and thus whether the receptors of the sensing unit of the detachable chip 11 can form a specific binding with the target object can be known.

The complex sensing element 13, 23 is disposed in the frame 3, and a value is read by an exterior spectrometer. The disposing method may include but is not limited to bonding, riveting, screwing, welding, scarfing and articulating. The disposing method of this embodiment is scarfing. The frame 3 has a scarfing column 31 to combine the scarfing hole 122, 222 of the base element with a through hole 12, 22 for use. The number of the through holes of the base element 12, 22 and the number of the sets of the complex sensing element 13, 23 may depend on the user's needs. As shown in FIG. 4(a) and FIG. 4(b), when the number of samples is 48, 6 sets of the complex sensing element 23 with the base element having 8 through holes 22 may be used. When the number of samples is 96, 12 sets of the complex sensing element 23 with the base element having 8 through holes 22 may be used. Unlike conventional 96-well plates, regardless of the number of samples, an entire 96-well plate is used. On the other hand, when the number of samples is large but the quantity of the individual sample is small, or when samples are expensive, 1 set of the complex sensing element with the base element having 384 through holes may be used. Therefore, a large number of samples may be handled at a time, and the amount of samples may be reduced. The value is a wavelength, and the read wavelength ranges from 300-700 nm. The read wavelength range will vary with the diameter of each metal nanoparticle (or the thickness of the metal layer) and the material of the metal nanoparticles. For example, when the average diameter of each metal nanoparticle is 5 nm-20 nm, the read wavelength will range from 400 nm-650 nm. When the total thickness of the metal layer is controlled at 3 nm, the formed gold nanoparticles will mainly range from 510 nm-540 nm, and the formed gold and silver alloy nanoparticles will mainly range from 410 nm-490 nm. The sensing device of the present invention has the same length and width as general experimental plates on the market, and therefore it is suitable for any instrument using the general experimental plates such as a spectrometer and an automatic microplate washer, wherein the spectrometer may be an ELISA reader.

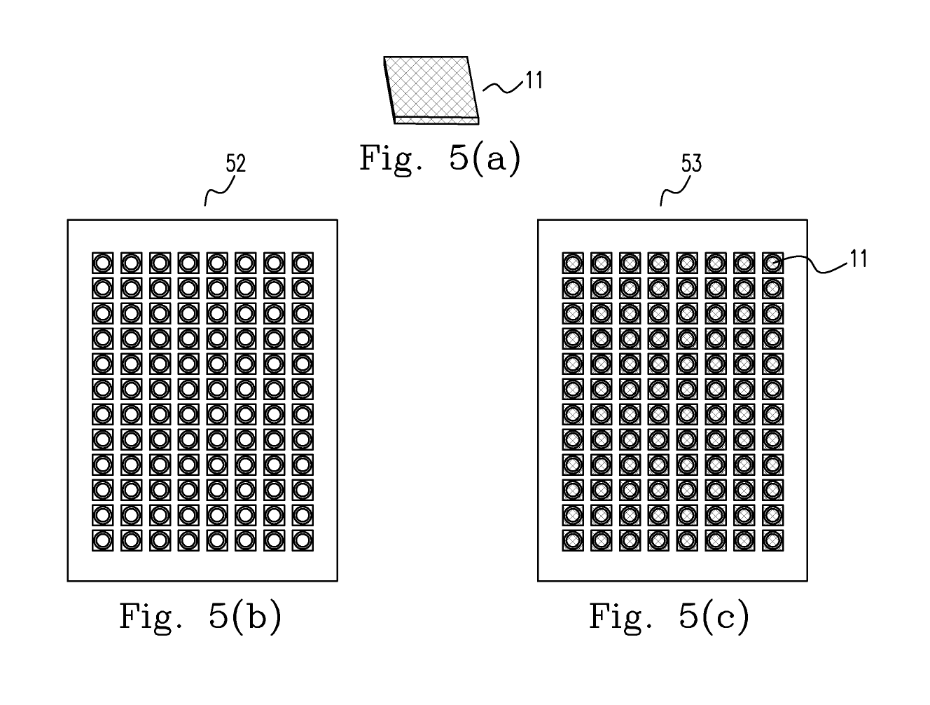

As shown in FIG. 5(a)-FIG. 5(c), another preferred embodiment of the present invention is a sensing device. The sensing device includes a detachable chip 11 and a base element with a through hole 52, wherein the detachable chip 11 includes a nanoparticle unit. The detachable chip 11 is detachably arranged on the base element 52 to close the through hole at one end of the base element 52 to form a complex sensing element 53. The number of the through holes of the base element with a through hole 52 is an integer between 1-384. 1-384 detachable chips 11 may be detachably arranged on the base element 52. The sensing device of this embodiment is suitable for any instrument using general experimental plates without a frame. The instruments may be a spectrometer and an automatic microplate washer, wherein the spectrometer may be an ELISA reader.

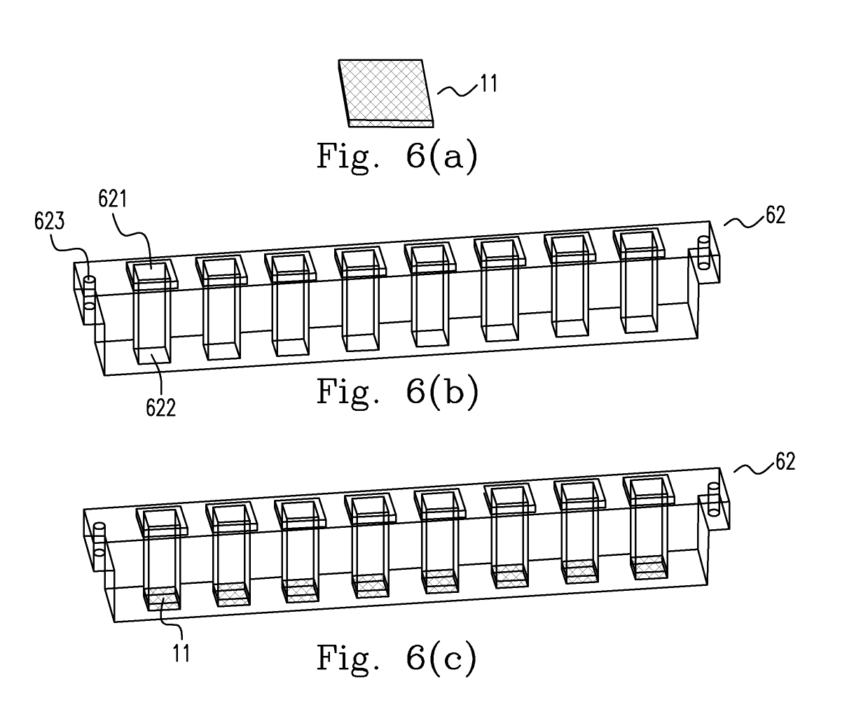

As shown in FIGS. 6(a)-6(c), the sensing chip carrier of another preferred embodiment of the present invention includes a carrier body 62 to carry a detachable chip 11 thereon, a chip accommodating part 621 disposed on the carrier body 62 to accommodate the detachable chip 11, and a detection light penetrating part 622 disposed on the carrier body 62 to allow a detection light to pass through the carrier body 62 and the detachable chip 11, wherein the detection light penetrating part 622 is a hollow part penetrating through the carrier body 62.

Compared with ELISA, the sensing method of the present invention does not require a secondary antibody linking a coloring enzyme and a substrate of the enzyme, and requires less time than ELISA. In addition, the amount of the target object required is also far less than ELISA, e.g. 20 μL. In addition, compared with the commercialized LSPR, the sensing method of the present invention can be used in a standard ELISA system that a general immunology laboratory is equipped with, and thus there is no need to purchase an expensive dedicated spectrometer. In addition, up to 384 samples can be operated simultaneously by using the sensing method of the present invention, and thus a high-throughput screening (HTS) effect is achieved. The sensing method of the present invention has absolute superiority regarding the time, price and convenience of use.

The sensing method of the present invention requires fewer steps, is label-free, is low-cost, requires minimal time, is coloring enzyme-free and can detect different antibodies and viruses.

The present invention is applicable to experimental developments such as immunoassay, chemical analysis and enzyme analysis. It is also applicable for the establishment of experimental procedures such as dynamics and temperature control. It is also applicable to antibody identification such as antibody/ligand affinity screening, epitope of monoclonal antibody screening, tumor cell screening and stage identification, anti-idiotypic antibody screening, antibody concentration measurements and fragment screening. It is also applicable to pre-clinical and clinical diagnostics such as bio-marker analysis and point of care.

### Experiment

1. The manufacture of microwave plasma nanoparticles:

This experiment follows the method disclosed in Taiwan Patent No. I404930. First, a gold film is sputtered on a glass substrate, and then the glass substrate is put into the microwave plasma to be processed, wherein the processing time is only 30 seconds. In the instant heating state under both the microwave and microwave plasma effects, gold nanoparticles are formed on the glass substrate, and the bottoms of the gold nanoparticles are coated with a layer of the glass structure. This greatly improves the adhesion between the gold nanoparticles and the substrate. A traditional heating method cannot produce this characteristic. It can be seen from this experiment that a microwave plasma heating method is low-cost, simple and fast. Nanoparticles are semi-embedded into the substrate, and thus have good adhesion and controllable size.

2. Detection of the substrate coating characteristic of the microwave plasma nanoparticles:

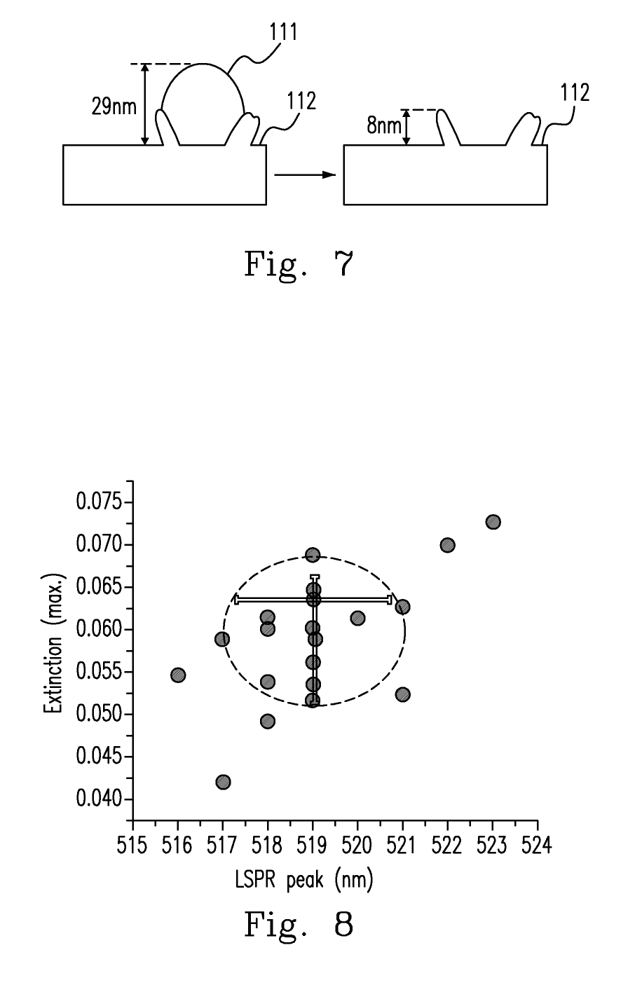

Please refer to FIG. 7. First, the gold nanoparticles 111 formed in the experiment are observed by an atomic force microscope (AFM), and it is found that the structure of the gold nanoparticles 111 is an island shaped structure. Then the substrate 112 is soaked in a nitric acid hydrochloride solution to remove the gold nanoparticles 111 from the substrate 112. The substrate 112 treated with the nitric acid hydrochloride solution is observed by using the atomic force microscope, and it is found that there are many cyclic structures remaining on the surface of the substrate 112. These cyclic structures are made of glass, which means that the bottoms of the gold nanoparticles 111 are now coated with a layer of glass. This is mainly because when nanoparticles are treated with microwave and microwave plasma, they reach a high temperature instantly and turn into nano-droplets in the high temperature state. The nano-droplets melt the glass substrate locally. Through gravity and capillary action, the molten glass gradually covers the surface of the nanoparticles and thus forms the island shape structure, wherein the gold nanoparticles 111 are semi-embedded into the substrate 112 in this experiment.

3. The manufacture of the sensing chip and the detection of reproducibility:

Please refer to FIG. 8, wherein 20 substrates are manufactured using the method of Experiment 1. The film thickness of the substrate is controlled at 2 nm and the processing time is 30 seconds. Optical absorption wavelengths of the 20 substrates are recorded after obtaining them. The optical absorption wavelengths of the 20 substrates fall in the range of 519±1.7 nm, which indicates a very small distribution range. Based on the optical distribution range, it can be seen that the 20 substrates have very high reproducibility, and thus they have substantial potential for the development of the disposable biological sensing chip.

4. Tests of the structural stability and surface oxidation effect of the sensing chip:

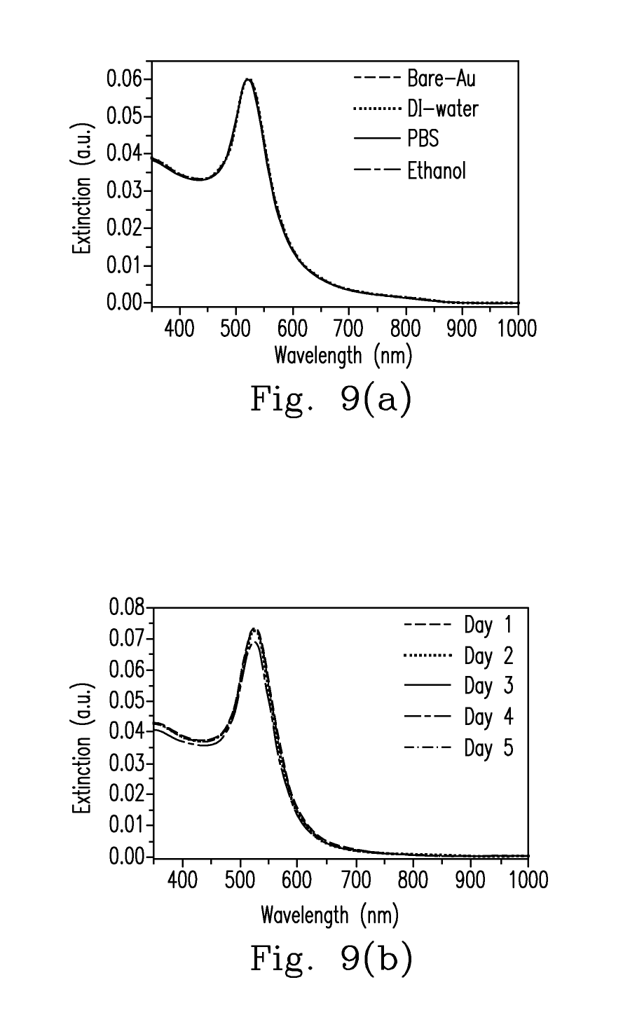

Please refer to FIGS. 9(a) and 9(b). This experiment further identifies the structural stability and surface oxidation of the substrate of the sensing chip. First, regarding the structural stability, it is feared that a sensing chip could fall off of the substrate or structurally deform when being soaked in a solution, thus resulting in an error of the optical signal during reading. In this experiment, the substrate is washed with ultrapure water, a PBS buffer solution and an ethanol solution. This does not cause significant optical changes, and the result shows that the nanostructure of the sensing chip of the present invention is very stable. Second, regarding the surface oxidation effect, due to the high surface activity of gold nanoparticles, the surface oxidation effect is also observed. It is found only that at the beginning, a thin and oxidized layer is generated on the surface of the gold nanoparticles, which causes a slight optical change. After one day, the gold nanoparticles trend to be stable and no more oxidation occurs. The result shows that the substrate of the present invention can be kept in storage for a long time.

5. Modifying the sensing chip of the present invention by (3-Aminopropyl)trimethoxysilane (APTMS) to carry out the detection of mercurous ion:

First, a hydrophilic modification is made to the substrate surface of the sensing chip of the present invention using oxygen plasma with a weaker energy, and then the substrate is soaked in the (3-Aminopropyl)trimethoxysilane (APTMS) solution such that (3-Aminopropyl)trimethoxysilane binds to the portions of the substrate without nanoparticles. Then, 4-carboxybenzo-15-crown-5-ether is linked to (3-Aminopropyl)trimethoxysilane to form a receptor. Finally, mercurous ion is added to react with the receptor in order to carry out the detection of the mercurous ion.

6. Modifying the sensing chip of the present invention by (3-Aminopropyl)trimethoxysilane (APTMS) to carry out the detection of streptavidin:

First, a hydrophilic modification is made to the substrate surface of the sensing chip of the present invention using oxygen plasma with a weaker energy, and then the substrate is soaked in the (3-Aminopropyl)trimethoxysilane (APTMS) solution such that (3-Aminopropyl)trimethoxysilane binds to the portions of the substrate without nanoparticles. Then, N-hydroxy-succinimide-biotin is added to link with (3-Aminopropyl)trimethoxysilane to form a receptor. Finally, streptavidin is added to react with the receptor in order to carry out the detection of streptavidin.

7. Modifying the sensing chip of the present invention by (3-Aminopropyl)trimethoxysilane (APTMS) to carry out the detection of an antigen or antibody:

First, a hydrophilic modification is made to the substrate surface of the sensing chip of the present invention using oxygen plasma with a weaker energy, and then the substrate is soaked in the (3-Aminopropyl)trimethoxysilane (APTMS) solution such that (3-Aminopropyl)trimethoxysilane binds to the portions of the substrate without nanoparticles. Then, Glutaraldehyde (GA) is added to form an imine bond with (3-Aminopropyl)trimethoxysilane. After that, an antibody (antigen) is added to form a receptor. Finally, the target antigen (antibody) is added to react with the receptor in order to carry out the detection of an antigen or antibody.

8. Further amplification of the signal of the sensing chip:

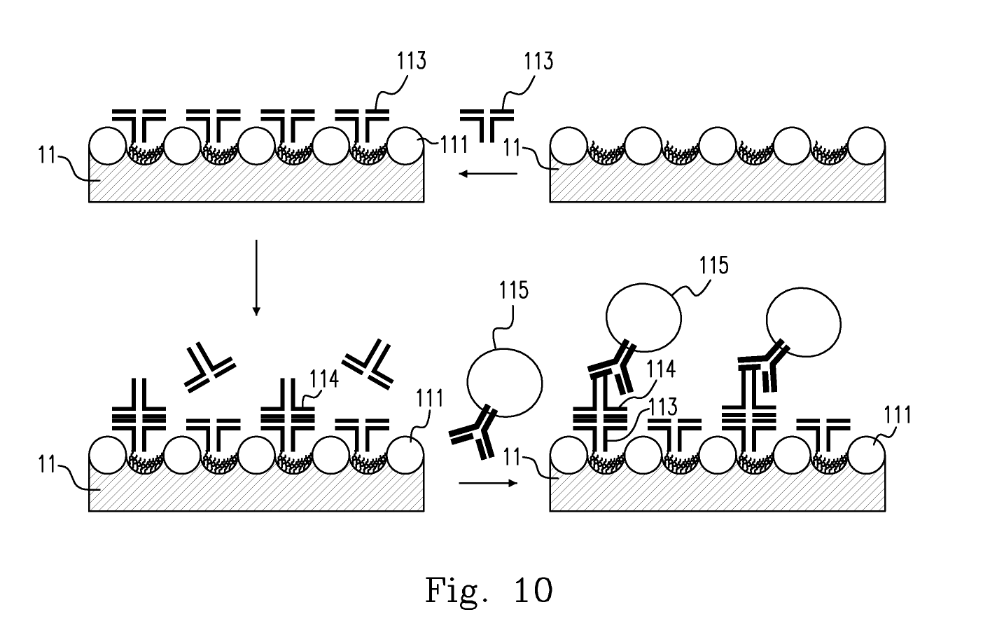

Please refer to FIG. 10. Unlike the traditional method of disposing an antibody molecule on the surface of the gold nanoparticles 111, in the sensing device of the present invention, (3-Aminopropyl)trimethoxysilane (APTMS) is disposed on the position of the substrate without the nanoparticles, then Glutaraldehyde (GA) is used as a linking molecule to link APTMS and the antibody 113, and next the antigen 114 is captured. After the antigen 114 is captured, in order to further observe the trace amount of a target object, the gold nanoparticles labeled with an antibody 115 are added to form a sandwich structure. Because there is a surface plasmon resonance coupling effect produced between the gold nanoparticles labeled with an antibody 115 and the gold nanoparticles semi-embedded into the substrate 111, a very high optical variation is generated. This causes the signal to be amplified for a thousand times, and thus the sensitivity reaches the level of picomole. Glutaraldehyde can also be used as a linking molecule to link APTMS and an antigen, followed by capturing the antibody. After the antibody is captured, in order to further observe the trace amount of the target object, the gold nanoparticles labeled with an antibody are added. Such a situation can also cause the signal to be amplified a thousand times, and thus the sensitivity reaches the level of picomole.

### Embodiments

1. A sensing method, comprising steps of providing a detachable chip including a substrate and a nanoparticle unit, wherein the substrate is made of a transparent material and the nanoparticle unit is arranged on the substrate and includes a plurality of nanoparticles spacedly disposed on the substrate; providing a base element with a through hole, and disposing the detachable chip detachably on the base element to close the through hole at one end of the base element to form a complex sensing element; providing a frame disposing therein the complex sensing element; disposing a first molecule to be adjacent to one of the plurality of nanoparticles; adding a target object to the complex sensing element to initiate a first specific binding between the first molecule and the target object; and disposing the complex sensing element in a spectrometer to obtain a value of a spectral signal of the plurality of spaced nanoparticles.
2. The sensing method of Embodiment 1, further comprising steps of adding a second molecule labeled with a luminant molecule to initiate a second specific binding between the target object and the second molecule.
3. The sensing method of Embodiments 1-2, further comprising steps of producing an electromagnetic field coupling between the luminant molecule and the plurality of nanoparticles when the target object exhibits the first specific binding with the first molecule and the second molecule exhibits the second specific binding with the target object.
4. The sensing method of Embodiments 1-3, wherein the plurality of nanoparticles are made of a metal and the metal is one selected from a group consisting of gold (Au), silver (Ag), palladium (Pd), platinum (Pt), chromium (Cr), cobalt (Co), molybdenum (Mo), copper (Cu), nickel (Ni), aluminum (Al), iron (Fe), magnesium (Mg), tin (Sn), titanium (Ti), thallium (Ta), iridium (Ir), an alloy thereof, and a combination thereof.
5. The sensing method of Embodiments 1-4, wherein the sensing method is used for at least one of qualitative and quantitative research of the target object.
6. The sensing method of Embodiments 1-5, wherein a variation of the value of a spectral signal comes from localized surface plasmon resonance (LSPR).
7. A sensing device, comprising a detachable chip; a base element having a through hole, wherein the detachable chip detachably arranged on the base element to close the through hole at one end of the base element to form a complex sensing element; and a frame disposing therein the complex sensing element.
8. The sensing device of Embodiment 7, wherein the detachable chip includes a substrate and a nanoparticle unit.
9. The sensing device of Embodiments 7-8, wherein the substrate is made of a transparent material.
10. The sensing device of Embodiments 7-9, wherein the nanoparticle unit is arranged on the substrate and includes a plurality of first nanoparticles spacedly disposed on the substrate.
11. A sensing method, comprising steps of causing a first molecule to be adjacent to one of a plurality of first nanoparticles spacedly disposed on a detachable chip; adding a target object to contact the first molecule; and measuring a spectral signal, wherein a variation of the spectral signal of the plurality of first nanoparticles occurs when the target object demonstrates a first specific binding with the first molecule.
12. The sensing method of Embodiment 11, wherein the first nanoparticles have a diameter ranged from 1-200 nm.
13. The sensing method of Embodiments 11-12, wherein every two adjacent first nanoparticles have a distance ranged from 1-100 nm.
14. The sensing method of Embodiments 11-13, wherein the first nanoparticles are made of a metal and the metal is one selected from a group consisting of gold (Au), silver (Ag), palladium (Pd), platinum (Pt), chromium (Cr), cobalt (Co), molybdenum (Mo), copper (Cu), nickel (Ni), aluminum (Al), iron (Fe), magnesium (Mg), tin (Sn), titanium (Ti), thallium (Ta), iridium (Ir), an alloy thereof, and a combination thereof.
15. The sensing method of Embodiments 11-14, wherein the sensing method is used for at least one of qualitative and quantitative research of the target object.
16. The sensing method of Embodiments 11-15, wherein the variation of the spectral signal comes from localized surface plasmon resonance (LSPR).
17. The sensing method of Embodiments 11-16, further comprising steps of adding a second nanoparticle labeled with a second molecule to initiate a second specific binding with the target object to amplify the variation of the spectral signal.
18. The sensing method of Embodiments 11-17, wherein the first nanoparticles are made of a metal and the metal is one selected from a group consisting of gold (Au), silver (Ag), palladium (Pd), platinum (Pt), chromium (Cr), cobalt (Co), molybdenum (Mo), copper (Cu), nickel (Ni), aluminum (Al), iron (Fe), magnesium (Mg), tin (Sn), titanium (Ti), thallium (Ta), iridium (Ir), an alloy thereof, and a combination thereof.
19. The sensing method of Embodiments 11-18, the sensing method is used for at least one of qualitative and quantitative research of the target object.
20. The sensing method of Embodiments 11-19, wherein the variation of the spectral signal comes from localized surface plasmon resonance (LSPR).

What is claimed is:

1. A sensing method, comprising steps of:
providing a detachable chip including a substrate and a nanoparticle unit, wherein the substrate is made of a transparent material and the nanoparticle unit is arranged on the substrate and includes a plurality of nanoparticles spacedly disposed on the substrate;
providing a base element with a through hole, and disposing the detachable chip detachably on the base element to close the through hole at one end of the base element to form a complex sensing element;
providing a frame disposing therein the complex sensing element;
disposing a first molecule to be adjacent to one of the plurality of nanoparticles;
adding a target object to the complex sensing element to initiate a first specific binding between the first molecule and the target object; and
disposing the complex sensing element in a spectrometer to obtain a value of a spectral signal of the plurality of spaced nanoparticles.

2. The sensing method according to claim 1, further comprising steps of:
adding a second molecule labeled with a luminant molecule to initiate a second specific binding between the target object and the second molecule.

3. The sensing method according to claim 2, further comprising steps of:
producing an electromagnetic field coupling between the luminant molecule and the plurality of nanoparticles when the target object exhibits the first specific binding with the first molecule and the second molecule exhibits the second specific binding with the target object.

4. The sensing method according to claim 1, wherein the plurality of nanoparticles are made of a metal and the metal is one selected from a group consisting of gold (Au), silver (Ag), palladium (Pd), platinum (Pt), chromium (Cr), cobalt (Co), molybdenum (Mo), copper (Cu), nickel (Ni), aluminum (Al), iron (Fe), magnesium (Mg), tin (Sn), titanium (Ti), thallium (Ta), iridium (Ir), an alloy thereof, and a combination thereof.

5. The sensing method according to claim 1, wherein the sensing method is used for at least one of qualitative and quantitative research of the target object.

6. The sensing method according to claim 1, wherein a variation of the value of a spectral signal comes from localized surface plasmon resonance (LSPR).

7. A sensing device, comprising:
a detachable chip;
a base element having a through hole, wherein the detachable chip detachably arranged on the base element to close the through hole at one end of the base element to form a complex sensing element; and
a frame disposing therein the complex sensing element.

8. The sensing device according to claim 7, wherein the detachable chip includes a substrate and a nanoparticle unit.

9. The sensing device according to claim 8, wherein the substrate is made of a transparent material.

10. The sensing device according to claim 8, wherein the nanoparticle unit is arranged on the substrate and includes a plurality of first nanoparticles spacedly disposed on the substrate.

11. A sensing method, comprising steps of:
causing a first molecule to be adjacent to one of a plurality of first nanoparticles spacedly disposed on a detachable chip;
adding a target object to contact the first molecule; and
measuring a spectral signal, wherein a variation of the spectral signal of the plurality of first nanoparticles occurs when the target object demonstrates a first specific binding with the first molecule.

12. The sensing method according to claim 11, wherein the first nanoparticles have a diameter ranged from 1-200 nm.

13. The sensing method according to claim 11, wherein every two adjacent first nanoparticles have a distance ranged from 1-100 nm.

14. The sensing method according to claim 11, wherein the first nanoparticles are made of a metal and the metal is one selected from a group consisting of gold (Au), silver (Ag), palladium (Pd), platinum (Pt), chromium (Cr), cobalt (Co), molybdenum (Mo), copper (Cu), nickel (Ni), aluminum (Al), iron (Fe), magnesium (Mg), tin (Sn), titanium (Ti), thallium (Ta), iridium (Ir), an alloy thereof, and a combination thereof.

15. The sensing method according to claim 11, wherein the sensing method is used for at least one of qualitative and quantitative research of the target object.

16. The sensing method according to claim 11, wherein the variation of the spectral signal comes from localized surface plasmon resonance (LSPR).

17. The sensing method according to claim 11, further comprising steps of:
adding a second nanoparticle labeled with a second molecule to initiate a second specific binding with the target object to amplify the variation of the spectral signal.

18. The sensing method according to claim 17, wherein the first nanoparticles are made of a metal and the metal is one selected from a group consisting of gold (Au), silver (Ag), palladium (Pd), platinum (Pt), chromium (Cr), cobalt (Co), molybdenum (Mo), copper (Cu), nickel (Ni), aluminum (Al), iron (Fe), magnesium (Mg), tin (Sn), titanium (Ti), thallium (Ta), iridium (Ir), an alloy thereof, and a combination thereof.

19. The sensing method according to claim 17, the sensing method is used for at least one of qualitative and quantitative research of the target object.

20. The sensing method according to claim 17, wherein the variation of the spectral signal comes from localized surface plasmon resonance (LSPR).
